(19) United States
(12) Patent Application Publication
(10) Pub. No.: US 2024/0199526 A1
(43) Pub. Date: Jun. 20, 2024

(54) METHOD FOR SYNTHESIZING COMPOUND IN WHICH ACETOXYLATION REACTION IS APPLIED TO LACTIC ACID DERIVATIVE

(71) Applicants: HYUNDAI MOTOR COMPANY, SEOUL (KR); KIA CORPORATION, SEOUL (KR); RESEARCH & BUSINESS FOUNDATION SUNGKYUNKWAN UNIVERSITY, SUWON-SI (KR)

(72) Inventors: Jong Geun Sung, Hwaseong-si (KR); Sung Wan Jeon, Suwon-si (KR); Do Hyun Ryu, Suwon-si (KR); Dong Guk Nam, Seoul (KR)

(21) Appl. No.: 18/202,571

(22) Filed: May 26, 2023

(30) Foreign Application Priority Data
Dec. 20, 2022 (KR) 10-2022-0178886

(51) Int. Cl. C07C 67/03; C07C 67/54

(52) U.S. Cl. CPC C07C 67/03; C07C 67/54

(57) ABSTRACT

A synthesis method according to the present disclosure includes preparing a mixture by mixing a lactic acid derivative, alkyl acetate, and methane sulfonic acid, and obtaining a final product by inputting alkyl acetate in the process of removing a distillate through fractional distillation of the mixture.

### CROSS REFERENCE TO RELATED APPLICATIONS

This application claims under 35 U.S.C. § 119(a) the benefit of priority to Korean Patent Application No. 10-2022-0178886 filed on Dec. 20, 2022, the entire contents of which are incorporated herein by reference.

### BACKGROUND

### (a) Technical Field

The present disclosure relates to a method for synthesizing a compound in which an acetoxylation reaction is applied to a lactic acid derivative.

### (b) Background Art

Methyl 2-acetoxy propionate (MAPA) and ethyl 2-acetoxy propionate (EAPA) may be synthesized by using acetic anhydride, acetic acid, and the like in a lactic acid derivative such as methyl lactate or ethyl lactate.

Methyl 2-acetoxy propionate (MAPA) and ethyl 2-acetoxy propionate (EAPA) are important intermediate substances for synthesizing a lactic acid-based acrylate such as methyl acrylate or ethyl acrylate.

The acrylate is used as a main raw material for acrylonitrile butadiene styrene (ABS) and styrene-acrylonitrile (SAN), which are polymers widely used as interior materials for vehicles, and it is a material used as a monomer of PAN fiber, which is a precursor of polyacrylonitrile (PAN) carbon fiber used in a hydrogen pressure container.

Meanwhile, since a conventional method for synthesizing the MAPA or the EAPA is a factor in lowering mass productivity due to the corrosiveness and exothermic reaction of the reactants, a method using trans-esterification using methyl acetate, ethyl acetate, and the like, which is more environmentally friendly and economical synthesis method, is emerging as an alternative.

Meanwhile, a method for synthesizing MAPA by trans-esterification of methyl acetate from lactide produced by NatureWorks is a high-temperature and high-pressure reaction and has a problem in that the yield is low.

Therefore, under the background as described above, there is a need for a method for synthesizing MAPA or EAPA that is eco-friendly and economical while having a higher yield.

### SUMMARY

The technical problem to be achieved by the present disclosure is to provide a method for synthesizing a compound that is eco-friendly and economical while having a high yield by applying an acetoxylation reaction to a lactic acid derivative.

The object of the present disclosure is not limited to the object mentioned above. The objects of the present disclosure will become more apparent from the following description, and will be realized by means and combinations thereof described in the claims.

A synthesis method according to the present disclosure includes a step of preparing a mixture by mixing a lactic acid derivative, alkyl acetate, and methane sulfonic acid, and a step of obtaining a final product by inputting alkyl acetate in the process of removing a distillate through fractional distillation of the mixture.

The lactic acid derivative may include ethyl lactate (EL) or methyl lactate (ML).

Alkyl acetate may include ethyl acetate (EA) or methyl acetate (MA).

In the step of preparing the mixture, the lactic acid derivative and alkyl acetate may be mixed at a molar ratio of 1:10 to 30.

In the step of preparing the mixture, the lactic acid derivative and methane sulfonic acid may be mixed at a molar ratio of 1:0.2 to 0.4.

The final product is characterized in that it is synthesized by trans-esterification.

The step of preparing the final product may be performed for 12 to 20 hours under reflux conditions at a temperature of 70 to 90° C.

The distillate may include alkyl acetate and ethanol.

The fractional distillation may be performed using a Dean-Stark Trap fractional distillation method.

The step of obtaining the final product is characterized by removing unreacted acetate.

The step of obtaining the final product may be performed by repeating steps of removing the condensed distillate and adding alkyl acetate.

In the step of adding alkyl acetate, the same amount of alkyl acetate as the removed distillate may be added at intervals of 2 to 4 hours.

The final product may be ethyl 2-acetoxy propionate (EAPA) or methyl 2-acetoxy propionate (MAPA).

The final product may have a conversion rate of the lactic acid derivative of 80 to 98%.

According to the present disclosure, EAPA or MAPA having a high yield can be obtained by using trans-esterification using economical ethyl acetate.

The esterification reaction of ethyl acetate used in the present disclosure facilitates control of the reaction temperature compared to the conventional synthesis method using acetic anhydride in lactide, and is environmentally friendly by generating ethanol, a low-toxic substance that is easily treated as a by-product.

The effects of the present disclosure are not limited to the above-mentioned effects. It should be understood that the effects of the present disclosure include all effects that can be inferred from the following description.

### BRIEF DESCRIPTION OF THE FIGURES

FIG. 1 is a flow chart showing a method for synthesizing a compound in which an acetoxylation reaction is applied to a lactic acid derivative according to the present disclosure.

FIG. 2 schematically shows a fractional distillation device used in the present disclosure.

### DETAILED DESCRIPTION

The above objects, other objects, features and advantages of the present disclosure will be easily understood through the following preferred embodiments related to the accompanying drawings. However, the present disclosure is not limited to the embodiments described herein and may be embodied in other forms. Rather, the embodiments introduced herein are provided so that the disclosed content may become thorough and complete, and the spirit of the present disclosure may be sufficiently conveyed to those skilled in the art.

The similar reference numerals have been used for similar elements while explaining each drawing. In the accompanying drawings, the dimensions of the structures are illustrated after being more enlarged than the actual dimensions for clarity of the present disclosure. Terms such as first, second, etc. may be used to describe various components, but the components should not be limited by the terms. The terms are used only for the purpose of distinguishing one component from another component. For example, a first component may be referred to as a second component, and similarly, the second component may also be referred to as the first component, without departing from the scope of rights of the present disclosure. The singular expression includes the plural expression unless the context clearly dictates otherwise.

In the present specification, terms such as “comprise”, “have”, etc. are intended to designate that a feature, number, step, operation, component, part, or a combination thereof described in the specification exists, but it should be understood that the terms do not preclude the possibility of the existence or addition of one or more other features, numbers, steps, operations, components, parts, or combinations thereof. Further, when a part of a layer, film, region, plate, etc. is said to be “on” other part, this includes not only the case where it is “directly on” the other part, but also the case where there is another part in the middle therebetween. Conversely, when a part of a layer, film, region, plate, etc. is said to be “under” other part, this includes not only the case where it is “directly under” the other part, but also the case where there is another part in the middle therebetween.

Unless otherwise specified, since all numbers, values, and/or expressions expressing quantities of components, reaction conditions, polymer compositions and formulations used in the present specification are approximate values reflecting various uncertainties of the measurement that arise in obtaining these values among others in which these numbers are essentially different, they should be understood as being modified by the term “about” in all cases. Further, when a numerical range is disclosed in this description, such a range is continuous, and includes all values from a minimum value of such a range to the maximum value including a maximum value, unless otherwise indicated. Furthermore, when such a range refers to an integer, all integers including from the minimum value to the maximum value including a maximum value are included, unless otherwise indicated.

The present disclosure relates to a method for synthesizing a compound in which an acetoxylation reaction is applied to a lactic acid derivative. Hereinafter, the present disclosure will be described in more detail with reference to the accompanying drawings.

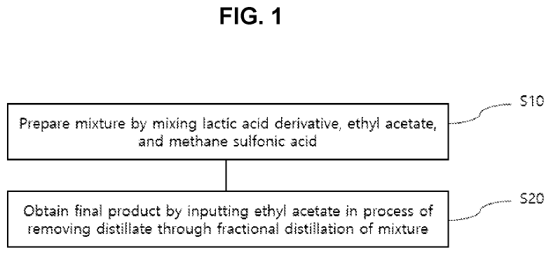

FIG. 1 is a flow chart showing a method for synthesizing a compound in which an acetoxylation reaction is applied to a lactic acid derivative according to the present disclosure.

Referring to FIG. 1, the synthesis method according to the present disclosure comprises a step S10 of preparing a mixture by mixing a lactic acid derivative, alkyl acetate, and methane sulfonic acid, and a step S20 of obtaining a final product by inputting alkyl acetate in the process of removing a distillate through fractional distillation of the mixture.

First, in the step S10, a mixture is prepared by mixing a lactic acid derivative, alkyl acetate, and methane sulfonic acid. The mixture may be mixed by a stirring process.

As the lactic acid derivative, ethyl lactate (EL) or methyl lactate (ML) may be used. In addition, alkyl acetate may include ethyl acetate (EA) or methyl acetate (MA).

In the step S10, the lactic acid derivative and alkyl acetate may be mixed at a molar ratio of 1:10 to 30. In the step S10, the lactic acid derivative and methane sulfonic acid may be mixed at a molar ratio of 1:0.2 to 0.4. Specifically, methane sulfonic acid may be used in an amount of 20 to 40 mol % based on the lactic acid derivative.

Subsequently, in the step S20, a final product is obtained by inputting alkyl acetate in the process of removing the distillate through fractional distillation of the mixture. In this case, the distillate may include alkyl acetate and ethanol. Here, alkyl acetate may include ethyl acetate (EA) or methyl acetate (MA).

In the step S20, the step may be performed for 12 to 20 hours under reflux conditions at a temperature of 70° ° C. to 90° C.

The reflux refers to a method for liquefying the volatile substances or solvents from above by installing a cooler or the like to prevent volatile substances or solvents from flying away while heating is continued so that they can flow again as a liquid.

In the step S20, unreacted acetate may be removed to obtain a final product.

Specifically, the step S20 may be performed by repeating steps of removing the condensed distillate and adding alkyl acetate. At this time, in the step of adding alkyl acetate, the same amount of alkyl acetate as the removed distillate may be added at intervals of 2 to 4 hours.

Accordingly, in the present disclosure, the overall molar ratio of reactants may be maintained through a process of continuously replenishing removed ethyl acetate.

Further, the fractional distillation used in the step S20 may be performed using a Dean-Stark Trap fractional distillation method. Specifically, the fractional distillation device used in the present disclosure may have a distillation tower installed in the upper portion thereof and have a fraction column using glass beads installed in the lower portion thereof.

The final product obtained in the step S20 may be ethyl 2-acetoxy propionate (EAPA) or methyl 2-acetoxy propionate (MAPA).

Specifically, EAPA may be prepared by Reaction Formula 1 below, and MAPA may be prepared by Reaction Formula 2 below.

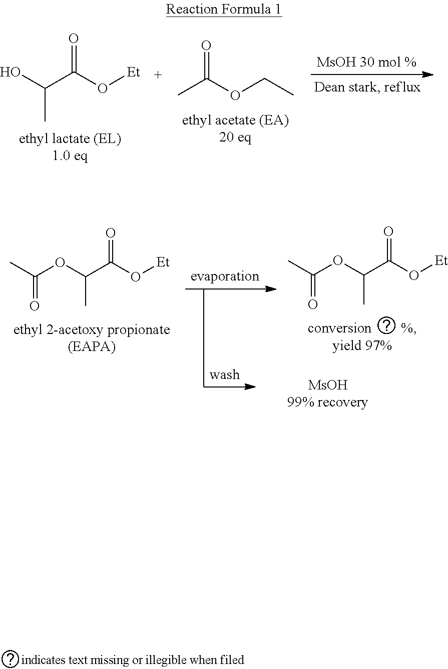

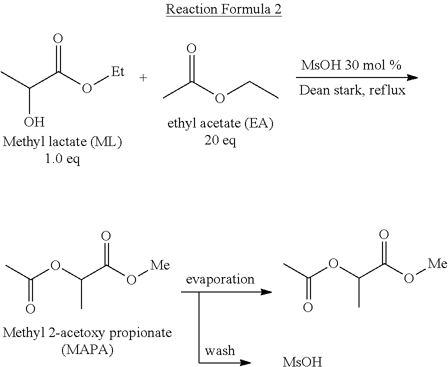

Ethyl 2-acetoxy propionate (EAPA) or methyl 2-acetoxy propionate (MAPA), which is the final product, may have a conversion rate of the lactic acid derivative of 80% to 98%.

Meanwhile, the present disclosure, as trans-esterification, is characterized in that refluxed ethyl acetate and ethanol, which is a side reactant, are continuously removed in certain amounts using a Dean Stark trap during the trans-esterification.

Therefore, the final product may be synthesized by trans-esterification.

Hereinafter, the present disclosure will be described in more detail through specific Examples. The following Examples are merely examples to help understanding of the present disclosure, and the scope of the present disclosure is not limited thereto.

### Preparation Example 1 (Catalyst Selection after Catalyst Screening)

First, in order to find an acid catalyst having a high yield in the synthesis method according to the present disclosure, a catalyst screening experiment was conducted to confirm the reaction yield of ethyl 2-acetoxy propionate (EAPA) under reaction conditions of an excessive amount of ethyl acetate.

The experiment for catalyst screening was carried out under reflux conditions for 8 hours using a seal tube with a 2 mmol scale of ethyl lactate (EL) and a molar reaction ratio of EL:ethyl acetate (EA) of 1:10. At this time, the reaction formula of prepared EAPA is shown in Reaction Formula 3 below.

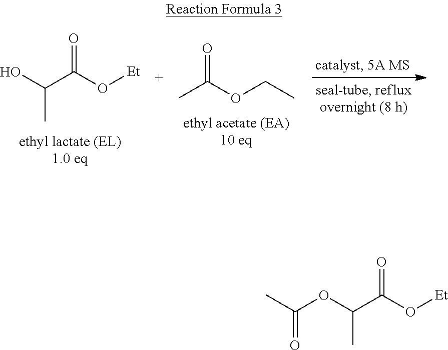

Lewis acid catalysts and Bronsted Acid catalysts corresponding to Table 1 below were used in experiments for catalyst screening. In addition, after applying the catalysts described below to the reaction, EAPA yields were measured, and the results are shown in Table 1 below.

TABLE 1

| Classification | Condition (20 mol %) | Adduct | Yield (%) |
|---|---|---|---|
| Lewis Acid | SnCl₂ | 5 A MS | Trace |
| Screening | MgCl₂ | 5 A MS | N.R |
| | CeCl₃ | 5 A MS | trace |
| | TMSCl | 5 A MS | trace |
| | BF3Et2O | 5 A MS | <5 |
| | FeCl₃ | 5 A MS | 11 |
| | AlCl₃ | 5 A MS | trace |
| Bronsted Acid | H₂SO₄ | 5 A MS | 52 |
| Screening | p-TSOH | 5 A MS | N.R |
| | HCl | 5 A MS | N.R |
| | MsOH | 5 A MS | 70 |
| | Acetic acid | 5 A MS | trace |
| | HNO₃ | 5 A MS | <10 |
| | TFA | 5 A MS | <5 |
| | Benzoic acid | 5 A MS | trace |
| | H₃PO₄ | 5 A MS | trace |

Referring to Table 1, the Lewis acid catalysts showed a reaction yield of up to 11% in FeCl₃, but did not show a significant effect compared to the Bronsted acid catalysts.

Further, yields of 50% or more were shown in sulfuric acid and methane sulfonic acid (MsOH), which are relatively strong acid catalysts. In particular, since the MsOH catalyst showed a yield of up to 70% in the above experiment, the MsOH catalyst was selected as a candidate catalyst.

### Preparation Example 2 (Selection of Catalyst Content)

Subsequently, in order to derive the optimal amount of MsOH, the catalysts were used in the amounts shown in Table 2 below in a state in which the EL:EA molar reaction ratio was fixed at 1:10 on the EL 2 mmol scale and the reflux reaction conditions were fixed, the catalysts were applied to the reaction, and then the reaction times, EL conversion rates, and EAPA yields were measured, and the measurement results are shown in Table 2 below.

TABLE 2

| | | Time | EL Conversion | EAPA |
|---|---|---|---|---|
| Classification | Catalyst | (hr) | Rate (%) | Yield (%) |
| 1 | MsOH (10 mol %) | 36 | 60 | 40 |
| 2 | MsOH (20 mol %) | 48 | 90 | 80 |
| 3 | MsOH (20 mol %) | 68 | 96 | 86 |
| 4 | MsOH (30 mol %) | 12 | 98 | 92 |
| 5 | MsOH (60 mol %) | 15 | 62 | 60 |

Referring to Table 2, the catalyst showed an EL conversion rate of 96% and an EAPA yield of 86% even when the reaction time was maintained up to 68 hours at a concentration of 20 mol % or less of MsOH. Therefore, it was confirmed that the target yield was not reached at a concentration of 20 mol % or less of MsOH despite the improvement of the reaction time.

Further, the activity of the side reaction rather than the forward reaction became more prominent due to the use of an excessive amount of catalyst in MsOH 60 mol %, and thus results of decreasing both of rather the EL conversion rate and the EAPA yield were shown.

Meanwhile, the EL conversion rate was shown to be 98% and the EAPA yield was shown to be 92% even though the reaction time was 12 hours in 30 mol % of MsOH, confirming the effect of increasing the MsOH concentration.

According to the above-mentioned results, an appropriate catalyst amount was confirmed to be 30 mol %.

### Preparation Example 3 (Adjustment of EA Content)

Subsequently, in order to derive the optimal reaction amount of EA, EA was used in the equivalent amount shown in Table 3 below in a state in which the catalyst amount was fixed at 30 mol % of MsOH on the EL 2 mmol scale and the reflux reaction conditions were fixed, the catalysts were applied to the reaction, and then EL conversion rates and EAPA yields according to the reaction time were measured, and the measurement results are shown in Table 3 below.

For reference, the above-mentioned experiment is an experiment to confirm the possibility of reducing the amount of EA used to improve economic feasibility, although 92% of EAPA yield was achieved in 12 hours when 10 eq of EA was used in the above-described catalyst screening experiment.

TABLE 3

| | EA | EL conversion rate (EAPA yield) (%) | | | |
|---|---|---|---|---|---|
| Classification | amount | 2 hr | 4 hr | 6 hr | 18 hr |
| 1 | 1.5 eq | 28 | 31 | 35 (29) | 64 (44) |
| 2 | 3.0 eq | 36 | 53 | 56 (51) | 58 (50) |
| 3 | 4.0 eq | 40 | 42 | 48 (45) | 58 (51) |
| 4 | 5.0 eq | 39 | 47 | 55 (52) | 53 (48) |
| 5 | 7.5 eq | 47 | 56 | 62 (59) | 61 (56) |

Referring to Table 3, it could be confirmed that the conversion rate and yield remained in the 60% range under 10 eq or less of EA.

According to the above-mentioned experimental results, the minimum reaction molar ratio of EL was determined to be 10 eq or more.

### Preparation Example 4 (EA Optimal Reaction Amount)

Subsequently, in order to derive the optimal reaction amount of EA, the method corresponding to Table 4 was applied to the reaction in a state in which the EA reaction amount was fixed at 20 eq and the catalytic amount was fixed at 30 mol % of MsOH on the EL 2 mmol scale, and the reflux reaction conditions were fixed, and then EL conversion rates and EAPA yields according to the reaction time were measured, and the measurement results are shown in Table 4 below. Here, with the scale improvement in mind, in order to suppress side reactions, the reaction was carried out in an EA reaction amount of 20 eq which is twice the previous EA reaction amount of 10 eq.

TABLE 4

| | | Time | EL conversion | EAPA |
|---|---|---|---|---|
| Classification | Method | (hr) | rate (%) | yield(%) |
| 1 | Seal-tube | 12 | 70 | 70 |
| 2 | Reflux only | 12 | 70 | 70 |
| 3 | Dean-Stark | 12 | 80 | 80 |
| 4 | Dean-Stark | 20 | 98 | 97 |
| 5 | Dean-Stark | 40 | 99 | 97 |

Referring to Table 4, the Reflux only method having only the Reflux condenser mounted thereon showed the same results as the experiment conducted in the existing seal tube.

Meanwhile, when Dean-stark, which can condense and remove ethanol that is a by-product, was used, improved EL conversion rates and EAPA yields could be confirmed compared to Seal tube or reflux only experimental method in the same 12-hour reaction.

Further, as a result of conducting the experiment by increasing the reaction time of the method using Dean-stark, an EL conversion rate of 98% and an EAPA yield of 97% were achieved in 20 hours.

However, it was confirmed that increasing the reaction time by 20 hours or more in the method using Dean-stark did not have a special effect.

### Preparation Example 5 (Checking Scale of Fractional Distillation Method)

Subsequently, in order to check the conditions capable of improving the synthesis scale when applying Dean-stark in the synthesis process, the following experiment was conducted using Dean-stark under the conditions of 20 eq of EA reaction amount and 30 mol % of catalytic amount of MsOH.

At this time, the reaction formula of prepared EAPA is shown in Reaction Formula 4 below.

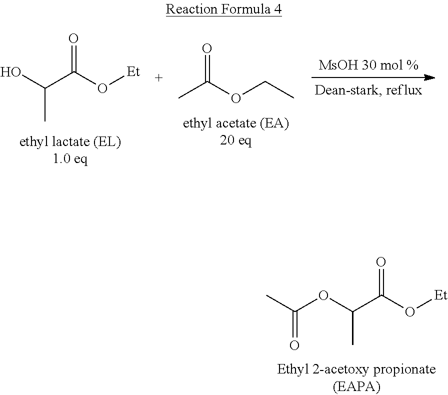

In the scale increase experiment, the EL conversion rates according to the reaction time were measured after applying the EA amounts corresponding to Table 5 below to the reaction, and the measurement results are shown in Table 5 below.

TABLE 5

| | | 24 hr EL | |
|---|---|---|---|
| | EL | conversion | |
| Classification | (mmol) | rate (%) | Remarks |
| 1 | 2 | 98 | 4 Å MS |
| 2 | 5 | 97 | 4 Å MS |
| 3 | 10 | 88 | 4 Å MS |
| 4 | 20 | 72 | 4 Å MS |
| 5 | 50 | 70 | 4 Å MS |
| 6 | 100 | 59 | 4 Å MS |
| 7 | 250 | 45 | 4 Å MS |
| 8 | 2 | 93 | w/o MS |
| 9 | 2 | 97 | w/o MS, EA refill |

Referring to Table 5, it is determined that the general Dean-stark utilization method, which separates/removes the distillate after condensation of a distillate at a reflux temperature, does not efficiently remove by-products (ethanol) as the scale improves.

Therefore, since the Dean-stark utilization method is a reflux reaction, the starting material, ethyl acetate, is also distilled together, resulting in loss.

Therefore, although the effect of ethyl acetate loss was insignificant on a small synthesis scale, it can be expected that the increased ethyl acetate loss over time lowers the synthesis yield on a large synthesis scale.

### Preparation Example 6 (Optimal Fractional Distillation Synthesis Method)

Subsequently, in order to select the optimal fractional distillation synthesis method, an experiment was conducted in which the removal time of distillate and the additional input time of ethyl acetate were adjusted under conditions using EL 0.1 mol scale, EA reaction amount 20 eq, catalytic amount MsOH 30 mol %, and Dean-stark.

In the experiment, the EL conversion rates according to the reaction time was measured after applying the method corresponding to Table 6 below to the reaction, and the measurement results are shown in Table 6 below.

TABLE 6

| | Time | Method 1 | Method 2 | Method 3 |
|---|---|---|---|---|
| Classification | (hr) | (conversion %) | (conversion %) | (conversion %) |
| 1 | 1 | 21 | 20 | — |
| 2 | 2 | 38 | 38 | — |
| 3 | 3 | 55 | 54 | 54 |
| 4 | 4 | 58 | 56 | — |
| 5 | 5 | 58 | 62 | — |
| 6 | 6 | 68 | 75 | 57 |
| 7 | 7 | 69 | 83 | — |
| 8 | 8 | 71 | 75 | 60 |
| 9 | 10 | 72 (side 10) | 75 | 71 (droplet) |
| 10 | 12 | 74 (side 14) | 60 (side 14) | 74 |
| 11 | 14 | — | — | 89 (droplet) |
| 12 | 24 | 61 (side 22) | 60 (side 21) | — |

Method 1: Remove distillate separated by 10 mL for each entry
Method 2: Remove distillate separated by 10 mL for each entry + add 10 mL of EA
Method 3: Continuously remove distillate by droplet method + add EA every 3 hours (add as much as removed)

Referring to Table 6, it can be seen that as the reaction time elapses and the reactant concentration changes, the effect of removing ethanol by Dean-stark decreases rapidly as the effect of the density difference between ethyl acetate and ethanol increases.

Therefore, referring to the above results, since the method of continuously removing the distillate as in (method 3) is effective, a method of using a distillation tower was proposed in the next test which is a 0.5 mol scale synthesis for scale improvement.

### Example 1

A distillate was continuously removed during the reaction process using 0.5 mol of ethyl lactate that is a lactic acid derivative, 20 eq of ethyl acetate, a catalytic amount 30 mol % of MsOH, and a distillation tower, and a final product was obtained through a compound synthesis method of adding the same amount of ethyl acetate removed at 3 hour intervals.

Specifically, 59 g of ethyl lactate, 800 mL of ethyl acetate, and 10 ml of methane sulfonic acid were mixed to carry out the reaction under reflux conditions while stirring the mixture at about 80 degrees Celsius for 3 hours. Additionally, while the reaction was being carried out under reflux conditions for about 14 hours, a distillate was continuously removed, and the same amount of ethyl acetate as the removed distillate was added at intervals of 3 hours. Subsequently, after the reaction was completed, a process of washing the catalyst used in the reaction with 300 mL of distilled water was repeated three times, and a process of neutralizing the reactants was performed using 400 mL of brine. Finally, a final product was obtained by performing a process of removing unreacted ethyl acetate using a rotary evaporator. Here, the prepared final product is ethyl 2-acetoxy propionate (EAPA) having a structure in which an ethyl group is attached to an ester group.

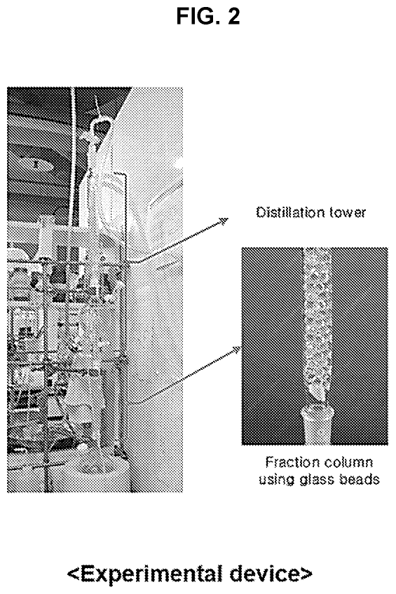

Further, the fractional distillation device shown in FIG. 2 was used in Example 1. Here, FIG. 2 schematically shows a fractional distillation device used in the present disclosure. Referring to FIG. 2, a distillation tower is installed in the upper portion of the fractional distillation device and a fraction column using glass beads is installed in the lower portion of the fractional distillation device.

### Example 2

A final product was obtained using a synthesis method under the same conditions as in Example 1 except that only a distillation tower was used without using a fraction column in the fractional distillation device.

### Example 3

A final product was obtained using a synthesis method under the same conditions as in Example 1 except that methyl lactate (ML) was used instead of ethyl lactate, as a lactic acid derivative, and methyl acetate was used instead of ethyl acetate. Here, the prepared final product is methyl 2-acetoxy propionate (MAPA) having a structure in which a methyl group is attached to an ester group.

After applying the method corresponding to the above Examples to the reaction, the EL conversion rates and ML conversion rates according to the reaction time were measured, and the measurement results are shown in Table 7 below.

Specifically, in Table 7, Examples 1 and 2 show measured EL conversion rates, and Example 3 shows measured ML conversion rates.

TABLE 7

| | | |
|---|---|---|
| Classification | Time (hr) | Conversion (%) |

| | | | |
|---|---|---|---|
| Example 2 | Distillation tower | 2.5 | 51 |
| | only (fraction | 7.5 | 64 |
| | column X) | 11 | 75 |
| | | 14 | 84 |
| | | 17 | 85 |
| | | 20 | 89 |
| Example 1 | Fraction | 3 | 40 |
| | column + | 6 | 60 |
| | Distillation tower | 9 | 75 |
| | | 13 | 91 |
| | | 16 | 93 |
| | | 17 | 98 |
| Example 3 | Fraction | 7 | 5 |
| | column + | 24 | 43 |
| | Distillation tower | 30 | 62 |
| | | 40 | 75 |
| | | 60 | 80 |

Referring to Table 7, the synthesis method according to Example 1 improved the ethanol removal efficiency by mounting a fraction column (glass beads) between the reaction flask and the distillation tower.

Accordingly, the synthesis method according to Example 1 showed an EL conversion rate of 98% and an EAPA selectivity of 99% with a reaction of 17 hours. Therefore, it could be found that the EL conversion rate was improved by about 10% compared to Example 2 using only the distillation tower.

Further, the synthesis method according to Example 3 was performed for a longer reaction time than the synthesis methods according to Examples 1 and 2, but the ML conversion rate was measured as low as about 80%.

This means that Example 3 has a lower starting material (ML) conversion rate than the results of Examples 1 and 2 in which EAPA was synthesized in EL.

Therefore, it was determined that the synthesis method according to Example 3 has high reactivity of methanol produced as a by-product, and due to this, many undesired products in the form of oligomers are produced instead of MAPA that is a target material.

Therefore, the esterification of ethyl acetate used in the present disclosure has an eco-friendly effect since the reaction temperature is easily controlled compared to the conventional synthesis method using acetic anhydride in lactide, and the esterification generates ethanol which is a low-toxic substance that is easily processed as a by-product.

Further, ethyl acetate used in the present disclosure is an inexpensive solvent that is frequently used commercially and has low toxicity.

Further, the present disclosure uses a method of performing simple distillation of ethyl acetate and ethanol in the separation/purification process after the reaction so that the production cost is low.

Further, the present disclosure uses relatively inexpensive methane sulfonic acid, and since methane sulfonic acid has high solubility in water, it can be easily removed. Therefore, in the experiment for the present disclosure, a reuse verification experiment was performed and it was confirmed that up to 98% of catalyst recovery is possible.

Therefore, the compound prepared by the synthesis method according to the present disclosure can be used to synthesize bio-based acrylate, and ABS, SAN, and carbon fiber using acrylate can be expected to have a carbon emission reduction effect as a material using biomass.

Although Examples of the present disclosure have been described with reference to the accompanying drawings, it will be understood that those skilled in the art to which the present disclosure pertains can implement the present disclosure in other specific forms without changing technical spirit or essential features of the present disclosure. Therefore, it should be understood that Examples described above are illustrative in all aspects and not restrictive.

1. A method for synthesizing a compound, the method comprising:
preparing a mixture by mixing a lactic acid derivative, alkyl acetate, and methane sulfonic acid; and
obtaining a final product by inputting alkyl acetate in a process of removing a distillate through fractional distillation of the mixture.

2. The method of claim 1, wherein the lactic acid derivative comprises ethyl lactate (EL) or methyl lactate (ML).

3. The method of claim 1, wherein alkyl acetate comprises ethyl acetate (EA) or methyl acetate (MA).

4. The method of claim 1, wherein in preparing the mixture, the lactic acid derivative and the alkyl acetate are mixed at a molar ratio of 1:10 to 30.

5. The method of claim 1, wherein in preparing the mixture, the lactic acid derivative and the methane sulfonic acid are mixed at a molar ratio of 1:0.2 to 0.4.

6. The method of claim 1, wherein the final product is synthesized by trans-esterification.

7. The method of claim 1, wherein preparing the final product is performed for 12 to 20 hours under reflux conditions at a temperature of 70° C. to 90° C.

8. The method of claim 1, wherein the distillate comprises alkyl acetate and ethanol.

9. The method of claim 1, wherein the fractional distillation is performed using a Dean-Stark Trap fractional distillation method.

10. The method of claim 1, wherein obtaining the final product comprises removing unreacted acetate.

11. The method of claim 1, wherein obtaining the final product is performed by repeating steps of removing the condensed distillate and adding alkyl acetate.

12. The method of claim 11, wherein in adding the alkyl acetate, the same amount of alkyl acetate as the removed distillate is added at intervals of 2 to 4 hours.

13. The method of claim 1, wherein the final product is ethyl 2-acetoxy propionate (EAPA) or methyl 2-acetoxy propionate (MAPA).

14. The method of claim 1, wherein the final product has a conversion rate of the lactic acid derivative of 80% to 98%.
